SEQUENCE LISTING (3 sequences)

SEQ ID NO: 1 - DNA, 45 bp, Artificial
  Single strand DNA oligonucleotide
tctgggtgacttggggctcaaggtatcaaggtaactatggcgggt

SEQ ID NO: 2 - DNA, 45 bp, Artificial
  Single strand DNA oligonucleotide
tccagtctttgtactttcttcatttccacctttgcccaagtcatc

SEQ ID NO: 3 - DNA, 39 bp, Artificial
  Single strand DNA oligonucleotide
tgatctgcgcgttaatgtgcggctccagcgcgccatagt

(19) United States
(12) Patent Application Publication
(10) Pub. No.: US 2007/0248702 A1
(43) Pub. Date: Oct. 25, 2007

(54) Use of CB2 receptors agonists for the treatment of Huntington's disease

(76) Inventor: Javier Fernandez-Ruiz, Tres Cantos (Madrid) (ES)

Correspondence Address: WINSTON & STRAWN LLP;PATENT DEPARTMENT, 1700 K STREET, N.W., WASHINGTON, DC, 20006, US

(21) Appl. No.: 11/642,615

(22) Filed: Dec. 19, 2006

Related U.S. Application Data
(63) Continuation of application No. PCT/IL05/00667, filed on Jun. 22, 2005.
(60) Provisional application No. 60/581,380, filed on Jun. 22, 2004.

(51) Int. Cl. A61K 36/13; A61P 25/28

(52) U.S. Cl. 424/770

(57) ABSTRACT

The present invention relates to ligands of the peripheral cannabinoid receptor CB₂, especially (+)-α-pinene derivatives, and to pharmaceutical compositions comprising these compounds, and to the use of such compounds for treatment and prevention of the onset of genetic neurodegenerative disorders, in particular Huntington's disease.

### CROSS-REFERENCE TO RELATED APPLICATIONS

This application is a continuation of International application PCT/IL2005/000667 filed Jun. 22, 2005, and claims the benefit of provisional application 60/581,380 filed Jun. 22, 2004, the entire content of each of which is expressly incorporated herein by reference thereto.

### FIELD OF THE INVENTION

The present invention relates to ligands of the peripheral cannabinoid receptor CB₂, especially (+)-α-pinene derivatives, to pharmaceutical compositions comprising these compounds, and to the use of such compounds for treatment and prevention of the onset of genetic neurodegenerative disorders, in particular Huntington's disease.

### BACKGROUND OF THE INVENTION

Huntington's disease (HD) is an adult-onset autosomal dominant neurodegenerative disorder caused by expanded CAG repeats in the huntingtin gene (Cattaneo E. et al., Trends Neurosci. 24: 182-8, 2001), which affects approximately 1 per 10,000 of the population in the West. In the United States alone, about 30,000 individuals have HD, and at least 150,000 others have a 50 percent risk of developing the disease. In 1983, Huntington's disease became the first major inherited disorder with an unidentified basic defect to be linked to a DNA marker. Huntingtin, the function of which remains incompletely defined, contains more than 3000 amino acids and is encoded by 10,366 bases on chromosome position 4p16.3.

The onset of Huntington's disease occurs at an average age of 35 to 40 years but can occur in people as young as two years of age or as old as 80 years of age. The onset is insidious and is characterized by abnormalities in coordination, movement, and behavior. Movement abnormalities include restlessness, mild postural abnormalities, and quick jerking movements of the fingers, limbs, and trunk. The movement abnormalities may be accompanied by substantial weight loss. Depression is common, and cognitive abnormalities and inappropriate behavior may develop. In contrast to the choreic movements typical of onset in adults, juvenile patients may exhibit rigidity, tremor, and dystonia. In the course of eight to fifteen years, the disorder progresses to complete incapacitation, with most patients dying of aspiration pneumonia or inanition.

This genetic disorder leads to the degeneration of neurons located primarily in the striatum, and scarcely affects striatal interneurons and dopaminergic afferents. The death of striatal projection neurons in HD may involve mitochondrial dysfunction, excitotoxicity, inflammation and oxidative stress (see Grunewald T. and Beal M. F., Ann. N.Y. Acad. Sci. 893: 203-13, 1999, for review). Several animal models, that reproduce some of the major events of the etiology of this disease, have been developed and exhibit most of the behavioral, histological and neurochemical hallmarks of HD (for review, see Brouillet E. et al., Prog. Neurobiol. 59: 427-68, 1999). These animal models have been used not only for elucidating the molecular mechanisms involved in the pathogenesis of the disease, but also to examine the potential of diverse compounds to alleviate motor symptoms and/or to slow the progress of neurodegeneration. Unfortunately, there is to date no efficacious pharmacotherapy for this disease and the search for novel compounds remains a major challenge for the future. Drug therapy of Huntington's disease is limited to the relief of symptoms, for example, reduction of severe chorea with antidopaminergic medication, improvement of hypokinetic rigidity with antiparkinsonian medication and the treatment of behavioral disturbances with neuroleptics and/or antidepressant agents. Moreover, these scarcely effective symptomatic therapies are not devoid of marked side effects. Ideally, treatment should improve functional capacity and arrest or delay striatal degeneration rather than simply suppress symptoms.

In the last two to three years, several studies have examined whether cannabinoid agonists can provide benefits for the treatment of HD, not only because of their anti-kinetic activities (Lastres-Becker I. et al., Synapse 44: 23-35, 2002 and J. Neurochem. 84: 1097-109, 2003), but also due to their neuroprotectant properties (for review, see Grundy R. I., Expert Opin. Investig. Drugs. 11: 1365-74, 2002; Mechoulam R. et al., Trends Mol. Med. 8: 58-61, 2002; Fernández-Ruiz J. et al., Prost. Leukot. Essent. Fatty Acids 66: 263-73, 2002; Fernández-Ruiz J. et al., Cannabinoids in neurodegeneration and neuroprotection. In: Cannabinoids as Therapeutics (Milestones in Drug Therapy), Miechoulam R. ed. Birkhäuser Verlag, Basel, 2005). Originally defined as any individual bioactive component of the plant cannabis, the term cannabinoids has come to encompass their endogenous counterparts and any synthetic compound that exerts most of its actions via the activation of the specific cannabinoid receptors. To date, two cannabinoid receptors have been cloned and characterized, cannabinoid receptor type 1 (CB₁) and cannabinoid receptor type 2 (CB₂), although additional receptors may exist. The CB₁ receptors are predominantly found in the central nervous system (CNS) and are responsible for the psychotropic effects of cannabinoids, whereas the CB₂ receptors are expressed mainly in the periphery on immune cells. The protective activities of cannabinoids in HD are believed to be mostly CB₁ receptor mediated because of its main localization in the CNS. The observation that CB₁ receptor density and signaling is affected in HD patients and transgenic mouse models of the disease also suggest that cannabinoids interacting with this receptor could be of therapeutic interest for HD (Lastres-Becker I. et al., Curr. Drug Target CNS Neurol. Disord. 2: 335-47, 2003).

Studies have been carried out in animal models of striatal injury generated either by administration of malonate or 3-nitropropionic acid (3-NP), two inhibitors of the mitochondrial complex II. These animal models reproduce efficaciously the deficiency in the mitochondrial complex found in HD patients and represent different but complementary aspects relating the neuronal death that occur during HD pathogenesis. In one of these models, rats with unilateral lesions of the caudate-putamen generated by local application of malonate, it was found that the administration of Δ⁹-THC apparently was pro-toxic, although the effects of this plant-derived and non-selective cannabinoid agonist were not dose-dependent (Lastres-Becker I. et al., Neuroreport 14: 813-6, 2003). This observation, together with the fact that SR141716, a selective CB₁ receptor antagonist, also enhanced malonate toxicity, indicates the possible ambiguous effects or overlapping of different mechanisms in the effects of cannabinoids in this rat model.

CB₁ is the cannabinoid receptor hitherto implicated in Huntington's disease and it is believed that highly selective CB₁ receptor agonists can produce neuroprotective effects in this disorder. No reports to date establish the efficacy of CB₂ selective agonists against this pathology.

U.S. Pat. No. 4,282,248 discloses both isomeric mixtures and individual isomers of pinene derivatives. Therapeutic activity, including analgesic, central nervous system depressant, sedative and tranquilizing activity, was attributed to the compounds, but the disclosure does not teach that these compounds bind to any cannabinoid receptor.

U.S. Pat. No. 5,434,295 discloses a family of novel 4-phenyl pinene derivatives, and teaches how to utilize these compounds in pharmaceutical compositions useful in treating various pathological conditions associated with damage to the central nervous system. U.S. Pat. No. 5,434,295 neither teaches nor suggests that any of the disclosed compounds are selective for peripheral cannabinoid receptors.

U.S. Pat. Nos. 6,864,291 and 6,903,137 disclose a family of bicyclic compounds, including (+){4-[4-(1,1-dimethylheptyl)-2,6-dimethoxy-phenyl]-6,6-dimethyl-bicyclo[3.1.1]hept-2-en-2-yl}-methanol (designated HU-308), as CB₂ specific agonists and exemplifies their use in the treatment of pain and inflammation, autoimmune diseases, gastrointestinal disorders and as hypotensive agents.

U.S. Pat. No. 5,434,295 discloses the neuroprotectice activity of pinene derivatives, including for the treatment of certain chronic degenerative diseases which are characterized by gradual selective neuronal loss. Genetic neurodegenerative diseases generally and Huntington's chorea specifically are not disclosed.

International patent application No. WO 03/064359 discloses that the CB₂ specific agonist HU-308 is useful in the treatment of Parkinson's disease (PD), as it reduces the extent of cell death in the substantia nigra of mice treated with the neurotoxin MPTP. However, WO 03/064359 does not teach or disclose that HU-308 is effective in treating HD.

Currently, no drug exists for preventing, alleviating or treating Huntington's disease. Thus, the present invention provides solutions to the long-felt unmet medical need for therapeutic means of intervening in or preventing onset of genetically determined neurodegeneration.

### SUMMARY OF THE INVENTION

It is an object of the present invention to provide methods for treating, alleviating or preventing the onset of a genetic neurodegenerative disorder, specifically Huntington's disease, by administering to an individual in need thereof a pharmaceutical composition comprising a therapeutically effective amount of a CB₂ selective agonist as an active ingredient.

This invention is based in part on the unexpected discovery that CB₂ specific agonists can inhibit neuronal degeneration and neurochemical deficit in an animal model mimicking Huntington's disease. The surprising involvement of CB₂ in the modulation of HD is further supported by the unforeseen up-regulation of the receptor in the lesioned areas of the brain.

According to certain embodiments, the CB₂ selective agonist used in the methods of the invention is a natural cannabinoid, plant derived or endogenous, or a synthetic cannabinoid, or metabolites and analogues thereof, typically selected from the group consisting of aminoalkylindoles, anandamides, 3-aroylindoles, aryl and heteroaryl sulfonates, arylsulphonamides, benzamides, biphenyl-like cannabinoids, cannabinoids optionally further substituted by fused or bridged mono- or polycyclic rings, pyrazole-4-carboxamides, eicosanoids, dihydroisoindolones, dihydrooxazoles, α-pinene derivatives, quinazolinediones, quinolinecarboxylic acid amides, resorcinol derivatives, tetrazines, triazines, pyridazines and pyrimidine derivatives, and isomers, analogues and derivatives thereof, as well as pharmaceutically acceptable salts, esters, solvates, prodrugs and polymorphs thereof.

According to additional embodiments, the CB₂ selective agonist used in the methods of the invention is a (+) or (−)-α-pinene derivative, or a mixture thereof.

According to a more preferred embodiment, the present invention provides a method of treating or alleviating Huntington's disease, comprising administering to an individual in need thereof a prophylactically and/or therapeutically effective amount of a pharmaceutical composition comprising as an active ingredient a compound of formula (I): 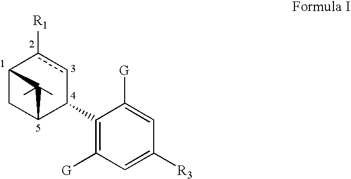
having a specific stereochemistry wherein C-5 is in the (S) configuration, the protons at C-1 and C-5 are cis in relation to one another and the protons at C-4 and C-5 are trans in relation to one another, wherein: the dashed line between C-2 and C-3 designates an optional double bond;
R₁ is selected from the group consisting of

(a) —R′ wherein R′ is a C₁-C₅ straight or branched chain alkyl;

(b) —OR″ wherein R″ is a hydrogen or a C₁-C₅ straight or branched chain alkyl optionally containing a terminal —OR′″ or —OC(O)R′″ moiety, wherein R′″ is a hydrogen or a C₁-C₅ straight or branched chain alkyl;

(c) -LN(R″)₂ wherein L is a C₁-C₅ straight or branched chain alkylene and at each occurrence R″ is as previously defined;

(d) -LX wherein L is as previously defined and X is halogen;

(e) -L^{a}C(O)N(R″)₂ wherein L^{a} is a direct bond or a C₁-C₅ straight or branched chain alkylene and R″ is as previously defined;

(f) -L^{a}C(O)OR″ or -L^{a}OC(O)R″ wherein La and R″ are as previously defined; and

(g) -LOR′″ wherein L and R′″ are as previously defined;

G is at each occurrence independently selected from the group consisting of hydrogen, halogen and —OR₂ wherein R₂ is a hydrogen or a C₁-C₅ straight or branched chain alkyl optionally containing a terminal —OR′″, —OC(O)R′″, C(O)OR′″, or —C(O)R′″ moiety wherein R′″ is as previously defined; and

R₃ is selected from the group consisting of

(a) a C₁-C₁₂ straight or branched chain alkyl;

(b) —OR″″ wherein R″″ is a straight or branched chain C₂-C₈ alkyl which can be optionally substituted at the terminal carbon atom by a phenyl group; and

(c) —(CH₂)ₙOR′″ wherein n is an integer of 1 to 7 and R′″ is as previously defined;

or a pharmaceutically acceptable salt, ester, solvate, polymorph or prodrug of the compound.

According to an exemplary embodiment, the present invention provides a method of treating or alleviating Huntington's disease, comprising administering to an individual in need thereof a prophylactically and/or therapeutically effective amount of a pharmaceutical composition comprising as an active ingredient a compound of formula (I) wherein there is a double bond between C-2 and C-3, R₁ is CH₂OH, G is OCH₃ and R₃ is 1,1-dimethylheptyl.

The present invention further encompasses the use for the preparation of a medicament for treating or alleviating Huntington's disease, of a CB₂ selective agonist typically selected from the group of aminoalkylindoles, anandamides, 3-aroylindoles, aryl and heteroaryl sulfonates, arylsulphonamides, benzamides, biphenyl-like cannabinoids, cannabinoids optionally further substituted by fused or bridged mono- or polycyclic rings, pyrazole-4-carboxamides, eicosanoids, dihydroisoindolones, dihydrooxazoles, α-pinene derivatives, quinazolinediones, quinolinecarboxylic acid amides, resorcinol derivatives, tetrazines, triazines, pyridazines and pyrimidine derivatives, and isomers, analogues and derivatives thereof, as well as pharmaceutically acceptable salts, esters, solvates, prodrugs and polymorphs thereof.

According to additional embodiments, the CB₂ selective agonist used for the preparation of a medicament is a (+) or (−)-α-pinene derivative, or a mixture thereof.

According to a further aspect, the present invention provides the use for the preparation of a medicament for treating or alleviating Huntington's disease, of a prophylactically and/or therapeutically effective amount of a compound of general formula (I): 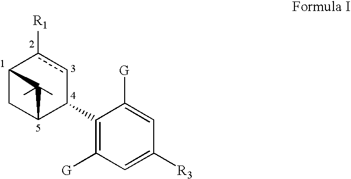
having a specific stereochemistry wherein C-5 is in the (S) configuration, the protons at C-1 and C-5 are cis in relation to one another and the protons at C-4 and C-5 are trans, in relation to one another, wherein:
the dashed line between C-2 and C-3 designates an optional double bond;
R₁ is selected from the group consisting of

(a) —R′ wherein R′ is a C₁-C₅ straight or branched chain alkyl;

(b) —OR″ wherein R″ is a hydrogen or a C₁-C₅ straight or branched chain alkyl optionally containing a terminal —OR′″ or —OC(O)R′″ moiety, wherein R′″ is a hydrogen or a C₁-C₅ straight or branched chain alkyl;

(c) -LN(R″)₂ wherein L is a C₁-C₅ straight or branched chain alkylene and at each occurrence R″ is as previously defined;

(d) -LX wherein L is as previously defined and X is halogen;

(e) -L^{a}C(O)N(R″)₂ wherein La is a direct bond or a C₁-C₅ straight or branched chain alkylene and R″ is as previously defined;

(f) -L^{a}C(O)OR″ or -L^{a}OC(O)R″ wherein La and R″ are as previously defined; and

(g) -LOR′″ wherein L and R′″ are as previously defined;

G is at each occurrence independently selected from the group consisting of hydrogen, halogen and —OR₂ wherein R₂ is a hydrogen or a C₁-C₅ straight or branched chain alkyl optionally containing a terminal —OR′″, —OC(O)R′″, C(O)OR′″, or —C(O)R′″ moiety wherein R′″ is as previously defined; and

R₃ is selected from the group consisting of

(a) a C₁-C₁₂ straight or branched chain alkyl;

(b) —OR″″ wherein R″″ is a straight or branched chain C₂-C₉ alkyl which can be optionally substituted at the terminal carbon atom by a phenyl group; and

(c) —(CH₂)ₙOR′″ wherein n is an integer of 1 to 7 and R′″ is as previously defined;

or a pharmaceutically acceptable salt, ester, solvate, polymorph or prodrug of the compound.

According to an exemplary embodiment, the present invention provides the use of a prophylactically and/or therapeutically effective amount of a compound of formula (I) wherein there is a double bond between C-2 and C-3, R₁ is CH₂OH, G is OCH₃ and R₃ is 1,1-dimethylheptyl, for the preparation of a medicament for treating or alleviating Huntington's disease.

The pharmaceutical compositions can contain in addition to the active ingredient conventional pharmaceutically acceptable thickeners, carriers, buffers, diluents, surface active agents, preservatives, excipients, and the like, all as well known in the art, necessary to produce physiologically acceptable and stable formulations.

The choice of the pharmaceutically inert additives, carriers, diluents, excipients and the like, will be determined in part by the particular active ingredient, as well as by the particular route of administration of the composition.

The pharmaceutical compositions can be administered by any conventional and appropriate route including oral, aerosol, parenteral, intravenous, intramuscular, intraperitoneal, subcutaneous, transdermal, intrathecal, rectal or intranasal.

The pharmaceutical compositions can be in a liquid, aerosol or solid dosage form, and can be formulated into any suitable formulation including, but not limited to, solutions, suspensions, micelles, emulsions, microemulsions, aerosols, powders, granules, sachets, soft gels, capsules, tablets, pills, caplets, suppositories, creams, gels, pastes, foams and the like, as will be required by the particular route of administration.

Prior to their use as medicaments for preventing, alleviating or treating an individual in need thereof, the pharmaceutical compositions can be formulated in unit dosage forms. The active dose for humans is generally in the range of from 0.01 mg to about 50 mg per kg body weight, and more preferably of about 0.1 mg to about 20 mg/kg, in a regimen of 1-4 times a day. However, it is evident to the man skilled in the art that the selected dosage of the active ingredient would be determined by the attending physician, according to the desired therapeutic effect, the method of administration, the patient's age, weight, contraindications, co-administration and combination with additional medications and the like. The administration of the composition of the present invention to a subject in need thereof, can be intermittent, or at a gradual or continuous, constant or controlled rate.

These and additional benefits and features of the invention will be better understood by those skilled in the art with reference to the following detailed description taken in conjunction with the figures and non-limiting examples.

### BRIEF DESCRIPTION OF THE FIGURES

The accompanying drawings, which are incorporated in and form a part of the specification, illustrate certain embodiments of the present invention, and together with the description serve to explain the principles of the invention. In the drawings:

FIGS. 1A, 1B and 1C show the GABA content in the caudate-putamen of rats with unilateral injections of malonate treated with the CB₁ receptor agonist, arachidonyl-2-chloroethylamide ACEA (FIG. 1A), the CB₂ receptor agonist HU-308 (FIG. 1B) or the major non-psychoactive constituent of Cannabis, CBD (FIG. 1C), and their respective controls of naïve animals (Control) and vehicle treated malonate-lesioned animals (Malonate).

FIG. 2 shows the mRNA levels for neuronal-specific enolase in the caudate-putamen of rats with unilateral injections of malonate treated with HU-308 or CBD, and their respective controls of naïve animals (Control) and vehicle treated malonate-lesioned animals (Vehicle).

FIG. 3 shows the GABA contents in the caudate-putamen of rats with unilateral injections of malonate treated with the CB₂ receptor agonist HU-308, the CB₂ receptor antagonist SR144528, or both, and their respective controls of naïve animals (Control) and vehicle treated malonate-lesioned animals (Vehicle).

FIGS. 4A and 4B show the immunostaining of CB₂ receptors in the caudate-putamen of rats with unilateral injections of malonate. FIG. 4A shows the lesioned side, whereas the FIG. 4B displays the non-lesioned side.

FIGS. 5A and 5B display the mRNA levels for SOD-1 (FIG. 5A) and SOD-2 (Figure B) in the caudate-putamen of rats with unilateral injections of malonate treated with HU-308 or CBD, and their respective controls of naïve animals (Control) and vehicle treated malonate-lesioned animals (Vehicle).

### DETAILED DESCRIPTION OF THE PREFERRED EMBODIMENTS

The present invention provides compositions and methods for alleviating, treating or preventing the onset of a genetic neurodegenerative disorder, specifically Huntington's disease. Huntington's disease appears to result from the premature death of certain systems of neurons in the brain and spinal cord. Neurons in various general regions of the brain are selectively vulnerable to cell death, with the most profound degeneration occurring in the corpus striatum (i.e., caudate nucleus and putamen). In addition, specific cell types within the corpus striatum are selectively vulnerable to loss.

In particular, the present invention provides pharmaceutical compositions comprising as an active ingredient CB₂ selective cannabinoid agonists and methods using the same for alleviating, treating or preventing the onset of Huntington's disease.

Typically, the CB₂ selective agonist is a natural, plant derived or endogenous, or a synthetic cannabinoid selected from the group consisting of aminoalkylindoles, anandamides, 3-aroylindoles, aryl and heteroaryl sulfonates, arylsulphonamides, benzamides, biphenyl-like cannabinoids, cannabinoids optionally further substituted by fused or bridged mono- or polycyclic rings, pyrazole-4-carboxamides, eicosanoids, dihydroisoindolones, dihydrooxazoles, α-pinene derivatives, quinazolinediones, quinolinecarboxylic acid amides, resorcinol derivatives, tetrazines, triazines, pyridazines and pyrimidine derivatives, and isomers, analogues and derivatives thereof, as well as pharmaceutically acceptable salts, esters, solvates, prodrugs and polymorphs thereof. More preferably, the CB₂ selective cannabinoid agonist is a α-pinene derivative, or a mixture of a (+) and (−)-α-pinene derivative, most preferably a (+)-α-pinene derivative.

Some of the compounds according to the invention can exist in stereoisomeric forms which are either enantiomers or diastereomers of each other. The invention relates to the enantiomers or diastereomers of the compounds or mixtures thereof. These mixtures of enantiomers and diastereomers can be separated into stereoisomerically uniform components in a known manner or synthesized a priori as separate enantiomers.

Definitions

To facilitate an understanding of the present invention, a number of terms and phrases are defined below.

As used herein, the term “central nervous system” (CNS) refers to all structures within the dura mater. Such structures include, but are not limited to, the brain and spinal cord.

As used herein, the term “CB” refers to cannabinoid receptors. CB₁ receptors are predominantly found in the CNS, whereas CB₂ receptors are predominantly found in the periphery on immune cells. Aside from these two receptors, evidence exists supporting the presence of yet uncloned cannabinoid receptors.

In the present invention, binding affinity is represented by the IC₅₀ value, namely the concentration of a test compound that will displace 50% of a radiolabeled agonist from the CB receptors. Preferred compounds display IC₅₀ value for CB₂ binding of 50 nM or lower, preferably of 30 nM or lower, more preferably of 10 nM or lower and most preferably of 1 nM or lower. “CB₂ specific or selective” denotes compounds with a ratio of CB₂/CB₁ binding affinity that is at least 10, preferably 20, more preferably 50 and most preferably 100 or greater. Preferably these ratios will be obtained for human CB₁ and CB₂ receptors. The selectivity toward CB₂, denoted CB₂/CB₁ affinity, is calculated as the IC₅₀ value obtained by the test compound for the displacement of the CB₁ specific radioligand divided by the IC₅₀ value obtained for the displacement of the CB₂ specific radioligand, i.e. the IC₅₀ CB₁/IC₅₀ CB₂. Some of the preferred compounds of the present invention do not necessarily share both properties, in other words some have an IC₅₀ ratio of 100 or greater for CB₂/CB₁ affinity and an IC₅₀ for CB₂ of only about 10 nM.

An agonist is a substance that mimics a specific ligand, for example a hormone, a neurotransmitter, or in the present case a cannabinoid, able to attach to that ligand's receptor and thereby produce the same action that the ligand produces. Though most agonists act through direct binding to the relevant receptor and subsequent activation, some agonists act by promoting the binding of the ligand or increasing its time of residence on the receptor, increasing the probability and effect of each coupling. Whatever the mechanism of action, all encompassed in the present invention, the net effect of an agonist is to promote the action of the original chemical substance serving as ligand. Compounds that have the opposite effect, and instead of promoting the action of a ligand, block it are receptor antagonists.

As used herein, the term “Huntington's disease” or “Huntington's chorea” refers to a progressive degenerative disease of the basal ganglia that is inherited as an autosomal dominant trait. Accurate animal models for Huntington's disease can be produced by generating lesions of the striatum or by treating with behavior-inducing agents.

Cannabinoids in Huntington's Chorea

Natural cannabinoids may be neuroprotectant in Huntington's disease (HD) (Romero J. et al., Pharmacol. Ther. 95: 137-152, 2002; Fernádndez-Ruiz J. et al., Cannabinoids in neurodegeneration and neuroprotection. In: Cannabinoids as Therapeutics (Milestones in Drug Therapy), Mechoulam R. ed. Birkhäuser Verlag, Basel, 2005, for review), an inherited neurodegenerative disorder characterized by progressive cell death, predominantly in the basal ganglia structures, which mainly results in motor abnormalities and cognitive decline. This proposal is noteworthy considering that, despite enormous progress in elucidating the molecular pathology of HD, since the first description of this disease in 1872, the progress for patients, in terms of having an effective pharmacotherapy with either symptomatic or protectant effects, has been poor. However, the capability of cannabinoids to reduce striatal degeneration in vivo remains to be demonstrated. In previous studies, the ability of Δ⁹-tetrahydrocannabinol (Δ⁹-THC), a plant-derived non-selective cannabinoid, to reduce the progress of neurodegeneration was examined in rat models of 3-NP induced striatal degeneration replicating the mitochondrial complex II deficiency characteristic of HD patients (Gu M. et al., Ann. Neurol. 39: 385-9, 1996; Sawa A. et al., Nat. Med. 5: 1194-8, 1999; Panov A. V. et al., Nat. Neurosci. 5: 731-6, 2002). A 9-THC was found to be neuroprotectant in rats systemically exposed to 3-nitropropionic acid (Lastres-Becker I. et al., Neuroreport 15: 2375-9, 2004). The neuroprotective potential of Δ⁹-THC in rats with striatal atrophy generated by unilateral injections of malonate was also examined (Lastres-Becker I. et al., Neuroreport 14: 813-6, 2003). Malonate is a complex II reversible inhibitor, which is known to produce neuronal death through activation of NMDA receptor (Beal M. F. et al., J. Neurochem. 61: 1147-50, 1993; Toulmond S. et al., Br. J. Pharmacol. 141: 689-97, 2004). However, the results in this last model were not conclusive, presumably because of overlapping between multiple mechanisms activated by this plant-derived cannabinoid.

Δ⁹-THC is a non-selective ligand to cannabinoid receptors. In the examples that will follow, the neuroprotective properties of cannabinoids in rats with striatal atrophy induced by unilateral application of malonate was re-examined by using more selective compounds, such as: (i) the CB₁ receptor agonist, arachidonyl-2-chloroethylamide (ACEA) (Hillard C. J. et al., J. Pharmacol. Exp. Ther. 289: 1427-33, 1999), (ii) the CB₂ receptor agonist, HU-308 (Hanus L. et al., Proc. Natl. Acad. Sci. USA. 96: 14228-33, 1999), and (iii) the plant-derived cannabinoid, cannabidiol (CBD), a compound with low affinity for cannabinoid receptors but having a considerable antioxidant capability (Mechoulam R. et al., J. Clin. Pharmacol. 42: 11S-19S, 2002). It was believed that CB₁ agonists would be effective in HD, and CB₂ agonists have not been tested for efficacy against this pathology.

As exemplified hereinbelow, it is now disclosed for the first time that the known CB₂ specific agonist HU-308, the full chemical name of which is (+){4-[4-(1,1-dimethylheptyl)-2,6-dimethoxyphenyl]-6,6-dimethyl-bicyclo[3.1.1]hept-2-en-2-yl}-methanol, also disclosed in WO 01/32169 as (+)4-[2,6-dimethoxy-4-(1,1-dimethylheptyl)phenyl]-6,6-dimethyl-bicyclo[3.1.1]hept-2-ene-2-carbinol, is particularly effective in the treatment of Huntington's disease. As disclosed in WO 03/064359, HU-308 binds human CB₂ receptors with an IC₅₀ of 13.3 nM and human CB₁ receptors with an IC₅₀ of 3600 nM, yielding a selectivity of about 270 fold for CB₂ binding affinity over CB₁.

As detailed in the Examples herein below, the neurodegeneration/neuroprotection, was measured by monitoring neurochemical deficits (γ-aminobutyric acid (GABA) and dopamine contents) in the basal ganglia, as well as mRNA levels for neuronal-specific enolase, a marker of neuronal integrity. Based on the results of this first series of experiments that for the first time suggested an unexpected important role for the cannabinoid CB₂ receptor subtype, experiments were carried out with the antagonist SR144528, which selectively blocks the effects mediated by the activation of this receptor subtype (Rinaldi-Carmona M. et al., J. Pharmacol. Exp. Ther. 284: 644-50, 1998). Immunocytochemical analyses were used to demonstrate the induction of CB₂ receptors in the lesioned areas and to confirm the involvement of the CB₂ receptors in the pathogenesis of the disease. Finally, possible involvement of non-receptor mediated mechanisms, such as antioxidant, anti-apoptotic or anti-inflammatory properties, was addressed.

Suitable CB₂ Selective Agonist Compounds

Suitable cannabinoid analogues are disclosed in U.S. Pat. No. 6,017,919 to Inaba et al. and in U.S. Pat. No. 6,166,066 to Makriyannis et al., the contents of which are hereby incorporated herein by reference in their entirety These compounds include acrylamide derivatives, benzamides, dihydroisoindolones, isoquinolinones, and quinazolinediones, as well as pentyloxyquinolines, dihydrooxazoles and non-classical cannabinoids in which the alkyl chain typically found in cannabinoids has been replaced with a monocyclic or bicyclic ring that is fused to the tricyclic core of classical cannabinoids.

United States Patent Applications Nos. 2004/0087590, 2004/0077851, 2004/0077649, 2003/0120094 and 2001/0009965 to Makriyannis et al., 2004/0034090 to Barth et al., 2003/0232802 to Heil et al., 2003/0073727 to Mittendorf et al., and 2002/0077322 to Ayoub, the contents of which are hereby incorporated herein by reference in their entirety, disclose a number of cannabinoid analogues suitable for use in the methods according to the present invention. These compounds include biphenyl and biphenyl-like cannabinoids, aminoalkylindoles, heterocyclic compounds including tetrazines, triazines, pyridazines and pyrimidine derivatives, 3-aroylindoles, aryl and heteroaryl sulfonates, arylsulphonamides and cannabinoids with a monocyclic, fused bicyclic, a bridged bicyclic or a bridged tricyclic side chain at the C-3 position of the phenyl ring of classical cannabinoids.

PCT Patent Application No. WO 03/091189 to Martin et al., incorporated herein by reference in its entirety, discloses a number of resorcinol derivatives suitable for use in the methods according to the present invention.

U.S. Pat. No. 4,208,351 to Archer et al. and PCT Patent Applications Nos. WO 01/28497 and WO 03/005960 to Makriyannis et al., WO 01/32169 to Fride et al., and WO 03/064359 and WO 03/063758 to Garzon et al., the contents of which are incorporated herein by reference in their entirety, disclose a number of classical and non-classical cannabinoid analogues suitable for use in the methods according to the present invention. These compounds include classical Δ⁹-THC type of compounds and bicyclic (−) and (+)-α-pinene derivatives.

In general, it has been possible to functionally differentiate between the R and S enantiomers of cannabinoid and cannabinoid-related compounds. The compounds HU-210 and HU-211 exemplify this. HU-210 is the (−)(3R,4R) enantiomer of the synthetic cannabinoid, 7-hydroxy-Δ⁶-tetrahydrocannabinol-1,1-dimethyl-heptyl. HU-211 is the (+)(3S,4S) enantiomer of this compound. In contrast to HU-210, HU-211 exhibits low affinity to the cannabinoid receptors and is thus non-psychotropic. In addition, it functions as a noncompetitive NMDA-receptor antagonist and as a neuroprotective agent, two properties absent in HU-210 (See, U.S. Pat. No. 5,284,867).

α-Pinene Compounds

The numbering of positions in the ring structure shown below is used to describe the α-pinene compounds used in the methods of the present invention. Positions 1, 4 and 5 are chiral centers. The stereochemistry of the preferred (+)-α-pinene derivatives is such that C-5 is the (S) configuration, the protons at C-1 and C-5 are cis in relation to one another and the protons at C-4 and C-5 are trans in relation to one another as shown in formula (II):

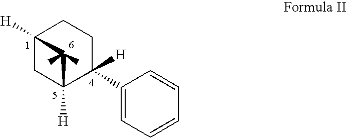

The stereochemistry of the (−)-α-pinene derivatives disclosed in the present invention is such that C-5 is in the (R) configuration, the protons at C-1 and C-5 are cis in relation to one another and the protons at C-4 and C-5 are trans in relation to one another.

Throughout this specification, certain compounds of the present invention can be referred to by capital letters followed by numbers, e.g. HU-308, rather than by their full chemical names. The alkyl substituents can be saturated or unsaturated (e.g. alkenyl, alkynyl), linear, branched or cyclic, the latter only when the number of carbon atoms in the alkyl chain is greater than or equal to three. When unsaturated, the hydrocarbon radicals can have one double bond or more and form alkenyls, or one triple bond or more and form alkynyls. Regardless of the degree of unsaturation, all of the alkyl substituents can be linear or branched.

OR represents hydroxyl or ethers, OC(O)R and C(O)OR represent esters, C(O)R represents ketones, C(O)NR₂ represents amides, NR₂ represents amines, wherein R is a hydrogen or an alkyl chain as defined above.

“Halogen” or “halo” means fluorine (—F), chlorine (—Cl), bromine (—Br) or iodine (—I) and if the compound contains more than one halogen (e.g., two or more variable groups can be a halogen), each halogen is independently selected from the aforementioned halogen atoms.

The term “substituted” or “optionally substituted” means that one or more hydrogens on the designated atom is replaced or optionally replaced with a selection from the indicated group, provided that the designated atom's normal valency under the existing circumstances is not exceeded. Combination of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

Pharmaceutically Acceptable Compounds

The present invention also includes within its scope solvates of compounds of formula (I) and salts thereof. “Solvate” means a physical association of a compound of the invention with one or more solvent molecules. This physical association involves varying degrees of ionic bonding, including hydrogen bonding. In certain instances the solvate will be capable of isolation. “Solvate” encompasses both solution-phase and isolatable solvates. Non-limiting examples of suitable solvates include alcohol solvates such as ethanolates, methanolates and the like. “Hydrate” is a solvate wherein the solvent molecule is water.

The term “polymorph” refers to a particular crystalline state of a substance, which can be characterized by particular physical properties such as X-ray diffraction, IR spectra, melting point, and the like.

In the present specification the term “prodrug” represents compounds which are rapidly transformed in vivo to parent compound of formula (I), for example by hydrolysis in the blood. Prodrugs are often useful because in some instances they can be easier to administer than the parent drug. They can, for instance, be bioavailable by oral administration whereas the parent drug is not. The prodrug can also have improved solubility compared to the parent drug in pharmaceutical compositions. All of these pharmaceutical forms are intended to be included within the scope of the present invention.

Certain compounds of the invention are capable of further forming pharmaceutically acceptable salts and esters. “Pharmaceutically acceptable salts and esters” means any salt and ester that is pharmaceutically acceptable, that is pharmacologically tolerated, and that has the desired pharmacological properties. Such salts, formed for instance by any carboxy group present in the molecule, include salts that can be derived from an inorganic or organic acid, or an inorganic or organic base, including amino acids, which is not toxic or otherwise unacceptable.

Pharmaceutically acceptable acid addition salts of the compounds include salts derived from inorganic acids such as hydrochloric, nitric, phosphoric, sulfuric, hydrobromic, hydriodic, phosphorous, and the like, as well as salts derived from organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate or galacturonate (Berge S. M. et al., J. of Pharmaceutical Science, 66: 1-19, 1977).

The acid addition salts of the basic compounds are prepared by contacting the free base form with a sufficient amount of the desired acid to produce the salt in the conventional manner. The free base form can be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention, such as for use as therapeutic agents for treating HD.

The base addition salts of the acidic compounds are prepared by contacting the free acid form with a sufficient amount of the desired base to produce the salt in the conventional manner. The free acid form can be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the present invention, such as for use as therapeutic agents for treating, preventing or alleviating HD.

Pharmacology

In the present specification and claims which follow the term “prophylactically effective” refers to the amount of compound which will achieve the goal of prevention of onset, reduction or eradication of the risk of occurrence of the disorder, while avoiding adverse side effects. The term “therapeutically effective” refers to the amount of compound that will achieve, with no or few adverse effects, alleviation, diminished progression or treatment of the disorder, once the disorder cannot be further delayed and the patients are no longer asymptomatic, hence providing either a subjective relief of a symptom(s) or an objectively identifiable improvement as noted by the clinician or other qualified observer. The compositions of the present invention are prophylactic as well as therapeutic and treating or alleviating the disease is explicitly meant to include preventing or delaying the onset of the disease.

The discovery of the HD gene in 1993 resulted in a direct genetic test to make or confirm a diagnosis of HD in an individual who is exhibiting HD-like symptoms or who have a family history of HD but is asymptomatic. Using a blood sample, the genetic test analyzes DNA for mutations in huntingtin by counting the number of CAG repeats. Individuals who do not have HD usually have 28 or fewer CAG repeats. Individuals with 29 to 34 CAG repeats will not most likely develop HD, but the next generation is at risk. The probability to develop HD is increased in individuals in the range of 35 to 39 CAG repeats, the next generation also being at risk, and individuals with 40 repeats or more are likely to develop HD. Moreover, there is an inverse relationship between the number of CAG repeats and the age of onset of symptoms. Identification of pre-symptomatic individuals at risk allows the prophylactic administration of the compositions of the invention to prevent or delay the onset of the disease.

The “individual” or “patient” for purposes of treatment includes any human or animal affected by any of the diseases where the treatment has beneficial therapeutic impact. Usually, the animal is a vertebrate such as a primate including chimpanzees, monkeys and macaques, a rodent including mice, rats, ferrets, rabbits and hamsters, a domestic or game animal including bovine species, equine species, pigs, sheeps, caprine species, feline species, canine species, avian species, and fishes.

Hereinafter, the term “oral administration” includes, but is not limited to, administration by mouth for absorption through the gastrointestinal tract (peroral) wherein the drug is swallowed, or for trans-mucosal absorption in the oral cavity by buccal, gingival, lingual, sublingual and oro-pharyngeal administration. Compositions for oral administration include powders or granules, suspensions or solutions in water or non-aqueous media, sachets, capsules or tablets. The oral composition can optionally contain inert pharmaceutical excipients such as thickeners, diluents, flavorings, dispersing aids, emulsifiers, binders, preservatives and the like.

The term “parenteral administration” as used herein indicates any route of administration other than via oral administration and includes, but is not limited to, administration by intravenous drip or bolus injection, intraperitoneal, intratechal, subcutaneous, or intra muscular injection, topical, transdermal, rectal, nasal administration or by inhalation.

Formulations for parenteral administration include but are not limited to sterile aqueous solutions which can also contain buffers, diluents and other suitable additives.

The compositions described herein are suitable for administration in immediate release formulations, and/or in controlled or sustained release formulations. The sustained release systems can be tailored for administration according to any one of the proposed administration regimes. Slow or extended-release delivery systems, including any of a number of biopolymers (biological-based systems), systems employing liposomes, and polymeric delivery systems, can be utilized with the compositions described herein to provide a continuous or long-term source of therapeutic compound(s).

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

The pharmaceutical compositions can contain in addition to the active ingredient conventional pharmaceutically acceptable carriers, diluents and excipients necessary to produce a physiologically acceptable and stable formulation. The terms carrier, diluent or excipient mean an ingredient that is compatible with the other ingredients of the compositions disclosed herein, especially substances which do not react with the compounds of the invention and are not overly deleterious to the patient or animal to which the formulation is to be administered. For compounds having poor solubility, and for some compounds of the present invention that are characteristically hydrophobic and practically insoluble in water with high lipophilicity, as expressed by their high octanol/water partition coefficient and log P values, formulation strategies to prepare acceptable dosage forms will be applied. Enabling therapeutically effective and convenient administration of the compounds of the present invention is an integral part of this invention.

The pharmaceutical compositions can be in a liquid, aerosol or solid dosage form, and can be formulated into any suitable formulation including, but not limited to, solutions, suspensions, micelles, emulsions, microemulsions, aerosols, ointments, gels, suppositories, capsules, tablets, and the like, as will be required for the appropriate route of administration.

Solid compositions for oral administration such as tablets, pills, capsules, soft gels or the like can be prepared by mixing the active ingredient with conventional, pharmaceutically acceptable ingredients such as corn starch, lactose, sucrose, mannitol, sorbitol, talc, polyvinylpyrrolidone, polyethyleneglycol, cyclodextrins, dextrans, glycerol, polyglycolized glycerides, tocopheryl polyethyleneglycol succinate, sodium lauryl sulfate, polyethoxylated castor oils, non-ionic surfactants, stearic acid, magnesium stearate, dicalcium phosphate and gums as pharmaceutically acceptable diluents. The tablets or pills can be coated or otherwise compounded with pharmaceutically acceptable materials known in the art, such as microcrystalline cellulose and cellulose derivatives such as hydroxypropylmethylcellulose (HPMC), to provide a dosage form affording prolonged action or sustained release. Coating formulations can be chosen to provide controlled or sustained release of the drug, as is known in the art.

Other solid compositions can be prepared such as suppositories or retention enemas, for rectal administration using conventional suppository bases such as cocoa butter or other glycerides. Liquid forms can be prepared for oral administration or for injection, the term including but not limited to subcutaneous, transdermal, intravenous, intraperitoneal, intrathecal, and other parenteral routes of administration. The liquid compositions include aqueous solutions, with or without organic cosolvents, aqueous or oil suspensions including but not limited to cyclodextrins as suspending agent, flavored emulsions with edible oils, triglycerides and phospholipids, as well as elixirs and similar pharmaceutical vehicles. In addition, the compositions of the present invention can be formed as aerosols, for intranasal and like administration. For administration by inhalation, the compounds of the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch. Topical pharmaceutical compositions of the present invention can be formulated as solution, lotion, gel, cream, ointment, emulsion or adhesive film with pharmaceutically acceptable excipients including but not limited to propylene glycol, phospholipids, monoglycerides, diglycerides, triglycerides, polysorbates, surfactants, hydrogels, petrolatum or other such excipients as are known in the art.

Pharmaceutical compositions of the present invention can be manufactured by processes well known in the art, e.g., by means of conventional mixing, dissolving, granulating, dry-mixing, direct compression, grinding, pulverizing, dragee-making, levigating, emulsifying, encapsulating, entrapping or lyophilizing processes.

Prior to their use as medicaments, the pharmaceutical compositions will generally be formulated in unit dosage forms. The active dose for humans can be determined by standard clinical techniques and is generally in the range of from 0.01 mg to about 50 mg per kg body weight, in a regimen of 1-4 times a day. The preferred range of dosage varies with the specific compound used and is generally in the range of from about 0.1 mg to about 20 mg per kg body weight. However, it is evident to one skilled in the art that dosages would be determined by the attending physician, according to the disease or disorder to be treated, its severity, the desired therapeutic effect, the duration of treatment, the method and frequency of administration, the patient's age, weight, gender and medical condition, concurrent treatment, if any, i.e. co-administration and combination with additional medications, contraindications, the route of administration, and the like. The administration of the compositions of the present invention to a subject in need thereof can be continuous, for example once, twice or thrice daily, or intermittent for example once weekly, twice weekly, once monthly and the like, and can be gradual or continuous, constant or at a controlled rate.

Effective doses can be extrapolated from dose-response curves derived from in vitro or animal model test systems. For example, an estimated effective mg/kg dose for humans can be obtained based on data generated from mice or rat studies, for an initial approximation the effective mg/kg dosage in mice or rats is divided by twelve or six, respectively.

Pharmaceutical compositions of the present invention can also include one or more additional active ingredients. The administration and dosage of such second agents is according to the schedule listed in the product information sheet of the approved agents, in the Physicians Desk Reference (PDR) as well as therapeutic protocols well known in the art.

When two or more active ingredients are administered to achieve the therapeutic goals of the present invention, co-administration can be in a unique dosage form for or in separate dosage forms for combined administration. Combined administration in the context of this invention is defined to mean the administration of more than one therapeutic in the course of a coordinated treatment to achieve an improved clinical outcome. Such combined administration can occur at the same time and also be coextensive, that is, occurring during overlapping periods of time. As used herein, co-administration is explicitly meant to include combined therapies that are administered individually or as a single composition. When administered individually, the separate therapeutic agents can be administered at substantially the same time or under separate regimens.

A further aspect of the present invention provides a method of treating or alleviating Huntington's disease, comprising administering to an individual in need thereof a prophylactically and/or therapeutically effective amount of a pharmaceutical composition comprising as an active ingredient a compound of formula (I): 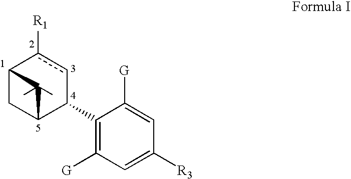
having a specific stereochemistry wherein C-5 is in the (S) configuration, the protons at C-1 and C-5 are cis in relation to one another and the protons at C-4 and C-5 are trans, in relation to one another, wherein:
the dashed line between C-2 and C-3 designates an optional double bond;
R₁ is selected from the group consisting of

(a) —R′ wherein R′ is a C₁-C₅ straight or branched chain alkyl;

(b) —OR″ wherein R″ is a hydrogen or a C₁-C₅ straight or branched chain alkyl optionally containing a terminal —OR′″ or —OC(O)R′″ moiety, wherein R′″ is a hydrogen or a C₁-C₅ straight or branched chain alkyl;

(c) -LN(R″)₂ wherein L is a C₁-C₅ straight or branched chain alkylene and at each occurrence R″ is as previously defined;

(d) -LX wherein L is as previously defined and X is halogen;

(e) -L^{a}C(O)N(R″)₂ wherein La is a direct bond or a C₁-C₅ straight or branched chain alkylene and R″ is as previously defined;

(f) -L^{a}C(O)OR″ or -L^{a}OC(O)R″ wherein La and R″ are as previously defined; and

(g) -LOR′″ wherein L and R′″ are as previously defined;

G is at each occurrence independently selected from the group consisting of hydrogen, halogen and —OR₂ wherein R₂ is a hydrogen or a C₁-C₅ straight or branched chain alkyl optionally containing a terminal —OR′″, —OC(O)R′″, C(O)OR′″, or —C(O)R′″ moiety wherein R′″ is as previously defined; and

R₃ is selected from the group consisting of

(a) a C₁-C₁₂ straight or branched chain alkyl;

(b) —OR″″ wherein R″″ is a straight or branched chain C₂-C₈ alkyl which can be optionally substituted at the terminal carbon atom by a phenyl group; and

(c) —(CH₂)ₙOR′″ wherein n is an integer of 1 to 7 and R′″ is as previously defined;

or a pharmaceutically acceptable salt, ester, solvate, polymorph or prodrug of the compound.

According to an exemplary embodiment, the present invention provides a method of treating or alleviating Huntington's disease, comprising administering to an individual in need thereof a prophylactically and/or therapeutically effective amount of a pharmaceutical composition comprising as an active ingredient a compound of formula (I) wherein there is a double bond between C-2 and C-3, R₁ is CH₂OH, G is OCH₃ and R₃ is 1,1-dimethylheptyl.

According to another aspect, the present invention provides the use of a prophylactically and/or therapeutically effective amount of a compound of formula (I) as described herein, for the preparation of a medicament for treating or alleviating Huntington's disease.

According to an exemplary embodiment, the present invention provides the use of a prophylactically and/or therapeutically effective amount of a compound of formula (I) wherein there is a double bond between C-2 and C-3, R₁ is CH₂OH, G is OCH₃ and R₃ is 1,1-dimethylheptyl, for the preparation of a medicament for treating or alleviating Huntington's disease.

The principles of the present invention will be more fully understood by reference to the following examples, which illustrate preferred embodiments of the invention and are to be construed in a non-limitative manner.

### EXAMPLES

The following examples are provided in order to demonstrate and further illustrate certain preferred embodiments and aspects of the present invention and are not to be construed as limiting the scope thereof. Most of the techniques used to prepare the animal model, testing the compounds and analyzing the outcome are widely practiced in the art, and most practitioners are familiar with the standard resource materials that describe specific conditions and procedures. However, for convenience, the following descriptions may serve as guidelines.

In the experimental disclosure which follows, the following abbreviations apply: N (normal); M (molar); mM (millimolar); kg (kilograms); g (grams); mg (milligrams); μg (micrograms); ng (nanograms); pg (picograms); ml (milliliters); μl (microliters); mm (millimeters); μm (micrometers); h (hours); min (minutes); ° C. (degrees Centigrade); i.p. (intraperitoneally); V (volt); mV (millivolt); nA (nanoampere); dpm (disintegrations per minute); SEM (standard error of the mean); and ns (not significant).

Unless otherwise stated, the statistical significance of the results was assessed by one-way or two-way analysis of variance (treatments×brain side), followed by the Student-Newman-Keuls test. A value of p<0.05 was considered significant.

### Example 1 Disease Induction and Treatment

The purpose of this study was to induce in a laboratory animal a pathology that mimics Huntington's disease in human. This animal model was then used to compare the efficacy of cannabinoids with various affinities and selectivities toward the CB₁ and CB₂ receptors.

Animals. Male Sprague-Dawley rats were housed in a room with controlled photoperiod (08:00-20:00 light) and temperature (23±1° C.). They had free access to standard food and water and were used at adult age (3 month-old; 300-400 g weight) for experimental purposes, all conducted according to European rules (directive 86/609/EEC). Each treatment group consisted of at least six animals.

Intrastriatal injection of malonate. Rats were injected stereotaxically (coordinates: +0.8 mm anterior, 2.9 mm lateral from the bregma, 4.5 mm ventral from the dura mater) into the left striatum with 2 M malonate in a volume of 1 μl and used for experimental analysis 48 h later.

Cannabinoid treatment. ACEA was purchased from Tocris (Biogen, Madrid, Spain), HU-308 was kindly provided by Pharmos (Rehovot, Israel), CBD was synthesized as previously described (Gaoni Y. and Mechoulam R., J. Am. Chem. Soc. 93: 217-24, 1971), and SR144528 was kindly provided by Sanofi-Synthelabo (Montpellier, France). All compounds were prepared in Tween 80-saline solution (1:16 volume per volume). The volume dosage for i.p. administration was 2 ml/kg body weight. The doses used for each experiment were selected from the previous studies on the pharmacological properties of these compounds.

In a first series of experiments, animals were i.p. administered with ACEA (3 mg/kg); HU-308 (5 mg/kg), CBD (5 mg/kg), or their corresponding vehicles 30 min before and 2 hours after the intrastriatal injection of malonate. Animals were killed 46 hours after the second cannabinoid injection and their brains were rapidly removed and frozen in 2-methylbutane cooled in dry ice, and stored until evaluation of the degree of malonate-induced striatal injury. The protocol and results concerning the neurochemical evaluation of the neuronal injury and the effect of the various types of cannabinoids are reported in Example 2. The protocol and results concerning the effect of the various types of cannabinoids on the mRNA levels of neuron specific enolase are reported in Example 3.

In a second experiment, SR144528, a CB₂ selective antagonist, was injected i.p. at a dose of 1 mg/kg according to the same schedule, with or without concomitant injection of HU-308 (5 mg/kg). Animals were also killed 46 hours after the second SR144528 and/or HU-308 injection and their brains were collected and processed as reported above. The protocol and results concerning the effect of SR144528 on the neuroprotective activity of HU-308 are reported in Example 4.

In a third experiment, malonate-injected and control rats were decapitated and their brains quickly and carefully removed, fixed in 4% paraformaldehyde in phosphate-buffered saline (PBS) for 24 h and embedded in paraffin. 4 μm-thick sections were obtained by a Leica microtome and mounted on glass slides to be used for immunohistochemical analyses. The protocol and results concerning the presence of CB₂ receptors in the brains of malonate-injected rats are reported in Example 5.

The first experiment was repeated to allow collection of animals' brains for analysis of the possible molecular mechanisms underlying HU-308 neuroprotective effect. The protocol and results concerning possible sites of action are reported in Example 6.

### Example 2 Neurochemical Evaluation of Neuronal Injury

The purpose of this study was to monitor the effect of various treatments administered in the animal model previously established as described in Example 1 by malonate intrastriatal injection in the rat on major neurotransmitters affected in Huntington's disease.

Sample preparation. Brains coronal slices (around 500 μm thick) were made at levels containing the substantia nigra, the globus pallidus and the caudate-putamen, according to Palkovits M. and Brownstein J. (Maps and Guide to Microdissection of the Rat Brain. Elsevier, 1988). Subsequently, the three structures were dissected and homogenized in 20-40 volumes of cold 150 mM potassium phosphate buffer, pH 6.8. Each homogenate was distributed for the analysis of the contents of GABA or dopainine and its major metabolite, 3,4-dihydroxyphenylacetic acid (DOPAC), by High Performance Liquid Chromatography (HPLC) coupled to electrochemical detection, according to previously described methods (Romero J. et al., Brain Res. 694: 223-32, 1995; González S. et al., Life Sci. 65: 327-36, 1999). An aliquot of each homogenate was used to analyze protein concentration (Lowry O. H. et al., J. Biol. Chem. 193: 265-75, 1951).

Analysis of GABA contents. This analysis was carried out by HPLC with electrochemical detection according to the procedure described by Smith S, and Sharp T. (J. Chromat. 652: 228-33, 1994). The aliquot of the homogenate used for the direct measurement of GABA content was diluted 1:2 with 0.4 N perchloric acid containing 0.4 mM sodium disulfite, 0.90 mM EDTA and 10 μg/ml β-aminobutyrate (BABA) as an internal standard. Following, samples were centrifuged for 3 min (15,000 g) and 50 μl of each supernatant removed and neutralized with 100 μl of 0.1 N NaOH. Samples were stored at 4° C. until analysis. This was performed by derivatization of GABA and BABA (β-aminobutyrate) through the addition of 15 μl of o-phthaldehyde (OPA)-sulfite solution (14.9 mM OPA, 45.4 mM sodium sulfite and 4.5% ethanol in 327 mM borate buffer, pH 10.4). Samples were allowed to react at room temperature for a period of 10 min. After this time, 20 μl of each reaction mixture (including derivatizated calibration standards composed of known concentrations of GABA and BABA) were injected into the HPLC system. The HPLC system consisted of the following elements. The pump was an isocratic Spectra-Physics 8810. The column was a RP-18 (Spherisorb ODS-2; 150 mm, 4.6 mm, 5 μm particle size; Waters, Mass., USA). The mobile phase, previously filtered and degassed, consisted of 0.06 M sodium dihydrogen phosphate, 0.06 mM EDTA and 20-30% methanol (pH 4.4) and the flow rate was 0.8 ml/min. The effluent was monitored with a Metrohm bioanalytical system amperometric detector using a glassy carbon electrode. The potential was 0.85V relative to an Ag/AgCl reference electrode with a sensitivity of 50 nA (approx. 2 ng per sample). The signal was recorded on a Spectra-Physics 4290 integrator. The approximate retention times for GABA and BABA were 8 and 16 min, respectively. The results were obtained from the peaks and calculated by comparison with the area under the corresponding internal standard peak. Values were expressed as μg/mg of protein.

Analysis of Dopamine and DOPAC contents. The contents of dopamine (DA) and its major intraneuronal metabolite, DOPAC, were analyzed using HPLC with electrochemical detection (Romero J. et al., Brain Res. 694: 223-32, 1995; González S. et al., Life Sci. 65: 327-36, 1999). Briefly, homogenates were diluted 1:2 in ice-cold 0.4 N perchloric acid containing 0.4 mM sodium disulfite and 0.90 mM EDTA. Dihydroxybenzylamine was added as an internal standard. The diluted homogenates were then centrifuged and the supernatants injected into the HPLC system, which consisted of a Spectra-Physics 8810 isocratic pump. The column was a RP-18 (Spherisorb ODS-2; 125 mm, 4.6 mm, 5 μm particle size; Waters, Mass., USA). The mobile phase consisted of 100 mM citric acid, 100 mM sodium acetate, 1.2 mM heptane sulphonate, 1 mM EDTA and 7% methanol (pH 3.9) and the flow rate was 0.8 ml/min. The effluent was monitored with a coulochemical detector (Coulochem II, ESA) using a procedure of oxidation/reduction (conditioning cell: +360 mV; analytical cell #1: +50 mV; analytical cell #2: −340 mV). The signal was recorded from the analytical cell #2, with a sensitivity of 50 nA (10 pg per sample), on a Spectra-Physics 4290 integrator and the results were given as area under the peaks. Values were expressed as ng/mg of protein.

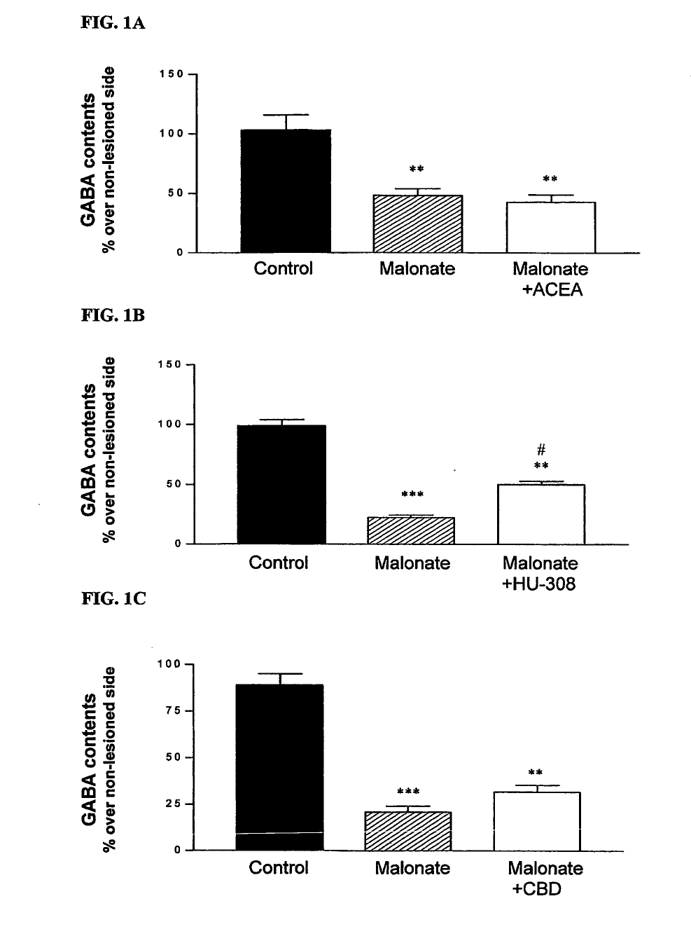

FIG. 1 shows the GABA content in the caudate-putamen of rats with unilateral injections of malonate treated with the CB₁ receptor agonist arachidonyl-2-chloroethylamide, ACEA (Panel A), the CB₂ receptor agonist HU-308 (Panel B) or the major non-psychotropic constituent of cannabis CBD (Panel C), and their respective controls of naïve animals (Control) and vehicle treated malonate-lesioned animals (Malonate). Values correspond to % of the lesioned side over the non-lesioned one for each individual, and are presented as means ±SEM of 6-8 determinations per group. Data were assessed by one-way analysis of variance followed by the Student-Newman-Keuls test (*p<0.05, **p<0.005, ***p<0.0005 vs. the controls; #p<0.05 vs. the malonate group).

Table 1 displays the GABA, dopamine and DOPAC contents in different basal ganglia of rats with unilateral injections of malonate treated with ACEA, HU-308 or CBD, and their respective controls as above described. Values correspond to % of the lesioned side over the non-lesioned one for each individual, and are presented as means ±SEM of 6-8 determinations per group. Data were assessed by one-way analysis of variance followed by the Student-Newman-Keuls test (*p<0.05, **p<0.005, ***p<0.0005 vs. the controls; #p<0.05 vs. the malonate group).

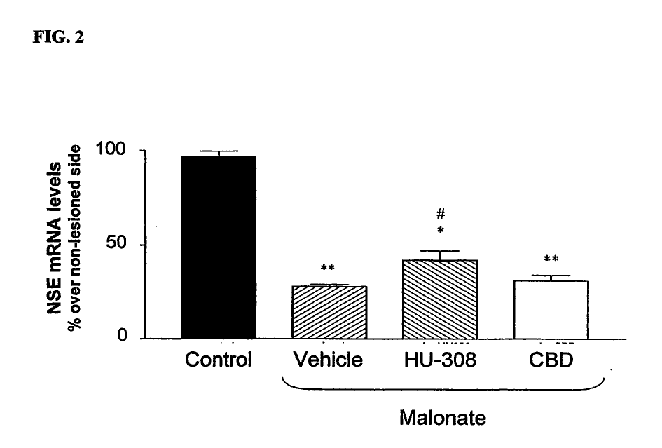

The neuroprotective effect of a CB₂ selective agonist in a model of HD. As expected, the application of malonate into the caudate-putamen produced marked reductions in GABA contents in this nucleus (FIG. 1) and also in structures receiving terminals of striatal output neurons, such as the globus pallidus (Table 1). The slight and non-significant reductions in GABA contents in the substantia nigra, another nucleus receiving terminals of striatal output neurons, is probably due to the dilution effect produced by the existence of other GABA-containing neurons that do not project from the caudate-putamen and, then, were not directly affected by malonate (Table 1). These effects are indicative of the death of striatal projection neurons, as has been previously reported by several authors (Moy L. Y. et al., J. Neurochem. 74: 1656-65, 2000; Zeevalk G. D. et al., Exp. Neurol. 176: 193-202, 2002). This was confirmed, as described herein below in Example 3, by a marked reduction in mRNA levels for neuronal-specific enolase, a marker of neuronal integrity, in the caudate-putamen (FIG. 2). Accompanying the death of striatal projection neurons caused by malonate, there was also a reduction in the contents of dopamine and its metabolite DOPAC in the caudate-putamen (Table 1), indicative of either dysfunction of nigrostriatal dopaminergic neurons that are under the influence of striatal output neurons (Calabresi P. et al., Prog. Neurobiol. 61: 231-65, 2000), or degeneration of dopaminergic terminals in the caudate-putamen by direct effect of malonate (Alfinito P. D. et al., J. Neurosci. 23: 10982-7, 2003).

TABLE 1

| | GABA contents | | DA contents | DOPAC contents |
|---|---|---|---|---|
| Treatments | Globus pallidus | Substantia nigra | Caudate-putamen | Caudate-putamen |
| Control | 97.2 ± 5.6 | 81.0 ± 9.2 | 87.5 ± 12.3 | 91.7 ± 9.5 |
| Malonate | 68.3 ± 12.7* | 78.5 ± 9.3 | 27.0 ± 4.6** | 41.6 ± 4.7** |
| Malonate + ACEA | 61.1 ± 12.2* | 92.1 ± 5.2 | 23.8 ± 5.4** | 44.9 ± 9.7** |
| Control | 112.0 ± 15.6 | 97.5 ± 7.2 | 104.3 ± 11.7 | 98.2 ± 12.6 |
| Malonate | 48.0 ± 18.8* | 89.8 ± 7.2 | 11.6 ± 7.6*** | 36.6 ± 9.1** |
| Malonate + HU-308 | 90.1 ± 13.4# | 93.8 ± 7.7 | 34.1 ± 5.2**# | 53.9 ± 5.7* |
| Control | 106.3 ± 11.9 | 104.2 ± 5.4 | 96.0 ± 7.9 | 96.2 ± 3.8 |
| Malonate | 62.0 ± 9.4* | 98.7 ± 8.4 | 7.6 ± 1.8*** | 14.0 ± 3.3*** |
| Malonate + CBD | 54.4 ± 8.2* | 86.3 ± 8.6 | 12.6 ± 4.9*** | 17.3 ± 5.5*** |

HU-308, a selective CB₂ receptor agonist, reduced malonate-induced GABA loss in the striatum (F(2,17)=94.21, p<0.0001; FIG. 1) and in the globus pallidus (F(2,18)=4.83, p<0.05; Table 1). HU-308 modified GABA contents only in the lesioned side (control: 1.91±0.13 μg/mg of protein; malonate: 0.76±0.07, p<0.001 vs. controls; malonate+HU-308: 1.12±0.08, p<0.05 vs. the other two groups; F(2,17)=36.81, p<0.0001), but not in the non-lesioned side (control: 1.90±0.06; malonate: 1.95±0.11; malonate+HU-308: 1.96±0.11; F(2,17)=0.112, ns), suggesting that its effects were neuroprotective (visible only in the lesioned side) rather than up-regulatory (visible in both sides). The same observations were made for the remaining regions and parameters. The data in the figures and tables are expressed as % in the lesioned side over the non-lesioned side for each individual. In the caudate-putamen, HU-308 also reduced the malonate-induced dopamine deficit, as reflected by a partial recovery in the contents for this neurotransmitter (F(2,18)=31,98, p<0.0001) and its metabolite DOPAC (F(2,19)=11.35, p<0.005) (Table 1).

In contrast to the results obtained with HU-308, ACEA, a selective CB₁ receptor agonist, did not influence neurochemical deficits induced by malonate application into the caudate-putamen and the other basal ganglia structures (Table 1 and FIG. 1). This outcome indicates that the activation of CB₁ receptors does not protect striatal projection neurons from the toxin-induced death. The same lack of neuroprotectant effects was also evident for CBD (Table 1 and FIG. 1), although CBD was able to slightly reduce the GABA depletion caused by malonate application in the caudate-putamen (FIG. 1). It is possible that this small effect of CBD is related to its ability to activate CB₂ receptors, for which it is a weak agonist, rather than produced by its antioxidant properties which are cannabinoid receptor-independent.

These results support the efficacy of CB₂ agonists for preventing neurochemical deficits associated with Huntington's disease.

### Example 3 In Situ Hybridization of Markers of Neuronal Integrity

The purpose of this study was to evaluate the presence of neuron-specific enolase (NSE) as a marker for neuroendocrine cells in the histologic diagnosis of Huntington's disease, as mimicked in the malonate injected rats. NSE is a generalized brain cell specific marker the level of which is decreased at lesioned sites.

Brain slicing. Coronal sections, 40 μm-thick, were cut in a cryostat, according to the Paxinos G. and Watson C. atlas (Rat brain in stereotaxic coordinates. Academic Press, London, 1986). Sections were thaw-mounted onto Superfrost Plus glass slides and dried briefly at 30° C. and stored at −80° C. until used.

Analysis of mRNA levels of NSE. Briefly, sections were fixed in 4% paraformaldehyde for 5 min and, after rinsing twice in PBS, were acetylated by incubation in 0.25% acetic anhydride, prepared in 0.1 M triethanolamine/0.15 M sodium chloride (pH 8.0), for 10 min. Sections were rinsed in 0.3 M sodium chloride/0.03 M sodium citrate, pH 7.0, dehydrated and delipidated by ethanol/chloroform series. For hybridization, the following synthetic probe of 45 nucleotides was used. This probe was selected based on the previously published sequence of rat NSE (SEQ ID No. 1; Katagiri T. et al., Mol. Brain Res. 19: 1-8, 1993).

SEQ ID No. 1

5′-TCTGGGTGAC TTGGGGCTCA AGGTATCAAG GTAACTATGG
CGGGT-3′

The oligonucleotide probe was labeled at the 3′-end with [³⁵S]-dATP using terminal deoxynucleotidyl-transferase. Sections were then hybridized with [³⁵S]-labelled oligonucleotide probes (7.5×10⁵ dpm per section), washed and exposed to X-ray film (βmax, Amersham) for 10 days, and developed (D-19, Kodak) for 6 min at 20° C. The intensity of the hybridization signal was assessed by measuring the grey levels in the films with a computer-assisted video densitometer. Adjacent brain sections were co-hybridized with a 100-fold excess of cold probe or with RNAse to assert the specificity of the signal.

FIG. 2 shows the mRNA levels for neuronal-specific enolase in the caudate-putamen of rats with unilateral injections of malonate treated with HU-308 or CBD, and their respective controls of naïve animals (Control) and vehicle treated malonate-lesioned animals (Vehicle). Values correspond to % of the lesioned side over the non-lesioned one for each individual, and are presented as means ±SEM of 6-8 determinations per group. Data were assessed by one-way analysis of variance followed by the Student-Newman-Keuls test (*p<0.005, **p<0.0005 vs. the controls; #p<0.05 vs. the malonate+vehicle or CBD groups).

As previously reported above, malonate injection caused a marked decrease in NSE mRNA levels in the lesioned side as compared to the non-lesioned side. The CB₂ agonist HU-308 partially prevented the malonate-induced reduction in mRNA levels for neuronal-specific enolase (F(3,21)=85.73, p<0.0001) (FIG. 2). While CBD appeared to be slightly neuroprotective when the levels of neurotransmitters were monitored, it seems to be totally inactive as far as neuronal integrity is concerned.

These results support the efficacy of CB₂ agonists for preventing at least in part the neuronal degeneration associated with Huntington's disease.

### Example 4 Impact of CB₂ Antagonist on Neuroprotective Activity

In the previous Examples, it was shown that out of the three types of cannabinoids tested, the CB₂ agonist was the only one able to prevent both the neurochemical deficit (FIG. 1 and Table 1) and the decrease in neuronal marker (FIG. 2). The goal of this study was to confirm that the neuroprotection observed in this model of Huntington's disease is mediated by the CB₂ receptor. For this purpose, the protective effect of HU-308 on levels of neurotransmitters was assessed in presence of the selective CB₂ antagonist SR144528. The experiment was carried out as described in Example 1.

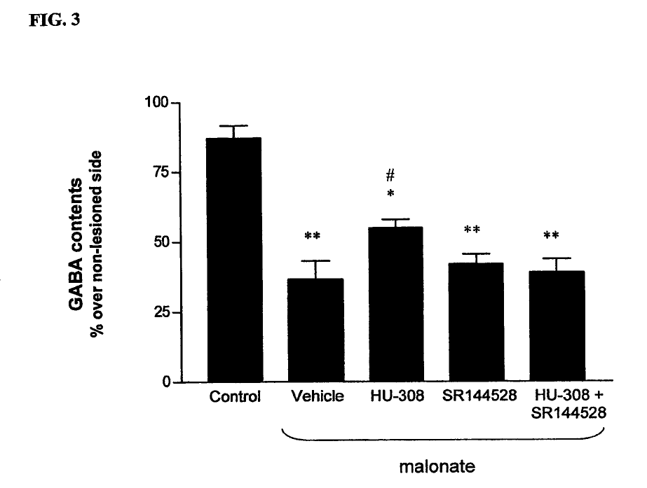

FIG. 3 shows the GABA contents in the caudate-putamen of rats with unilateral injections of malonate treated with the CB₂ receptor agonist HU-308, the CB₂ receptor antagonist SR144528, or both, and their respective controls of naïve animals (Control) and vehicle treated malonate-lesioned animals (Vehicle). Values correspond to % of the lesioned side over the non-lesioned one for each individual, and are presented as means ±SEM of 6-8 determinations per group. Data were assessed by one-way analysis of variance followed by the Student-Newman-Keuls test (*p<0.05, **p<0.005 vs. the controls; #p<0.05 vs. the other malonate groups).

The neuroprotective effect exerted by HU-308 in this rat model of HD is most likely related to the activation of CB₂ receptors. This can be concluded not only because HU-308 is a selective agonist for this receptor subtype, but also because the reduction by this agonist of malonate-induced GABA depletion in the caudate-putamen was completely reversed by the co-administration of the selective CB₂ receptor antagonist, SR144528. As shown in FIG. 3, malonate-induced GABA depletion in animals co-injected with HU-308 and SR144528 was similar to that of animals receiving vehicle and significantly different from that of animals receiving HU-308 alone (F(4,29)=15.78, p<0.0001).

These results, showing that the efficacy of a CB₂ selective agonist is blocked by a CB₂ selective antagonist, support the pivotal role of CB₂ in the mediation of neuroprotection in Huntington's disease.

### Example 5 Immunohistochemical Methods Supporting Involvement of CB₂ Receptors in HD

The goal of this study was to evaluate the involvement of the CB₂ receptor in the neuroprotective activity observed in the animal model of Huntington's disease. For this purpose the presence, relative amount and localization of CB₂ receptors was measured in the lesioned and non-lesioned areas of the brain.

Immunohistochemical staining of CB₂ receptors The protocol used for the immunohistochemical staining is basically the same as previously described (Tsou K. et al., Neuroscience 83: 393-411, 1998; Benito C. et al., J. Neurosci. 23: 11136-41, 2003) with slight modifications. Briefly, tissue sections were deparaffinizied and extensively washed in potassium phosphate-buffered saline (KPBS) (50 mM) and endogenous peroxidase was blocked by incubation in peroxidase-blocking solution (Dako, Denmark) for 20 min, at room temperature. In order to obtain a more efficient immunostaining, sections were subjected to an antigen retrieval procedure (Shi S. R. et al., J. Histochem. Cytochem. 49: 931-7, 2001). Briefly, sections were placed in a stainless steel pressure cooker containing a boiling solution (sodium citrate 0.01M, pH 10). After heating under pressure for 2 min, samples were removed and extensively washed in KPBS. Tissue sections were then incubated with the primary antibody (polyclonal anti-CB₂ receptor, 1:1500 dilution in KPBS, Affinity Bioreagents, USA). After 24 h incubation at 4° C., sections were washed in 50 mM KPBS and incubated with biotinylated goat anti-rabbit antibody (1:200), at room temperature for 1 h followed by avidin-biotin complex (Vector Elite, Burlingame, Calif., U.S.A.), according to the manufacturer's instructions. Visible reaction product was produced by treating the sections with 0.04% diaminobenzidine (DAB, Dako), 2.5% nickel sulfate and 0.01% H₂O₂, dissolved in 0.1 M sodium acetate. Sections were then dehydrated and sealed with cover slips. The observations and photography of the slides were done using a Nikon Eclipse E600 microscope and a Nikon Coolpix 4500 camera. Controls for the immunohistochemistry included the preabsorption and co-incubation of the antibodies with the corresponding immunogenic proteins (CB₂, fusion protein against amino acids 1-33 of human-CB₂ at 5 Hg/ml) and incubation in the absence of primary antibody. Adjacent sections to those employed in the immunohistochemical studies were used for hematoxilin-eosin and Nissl stainings.

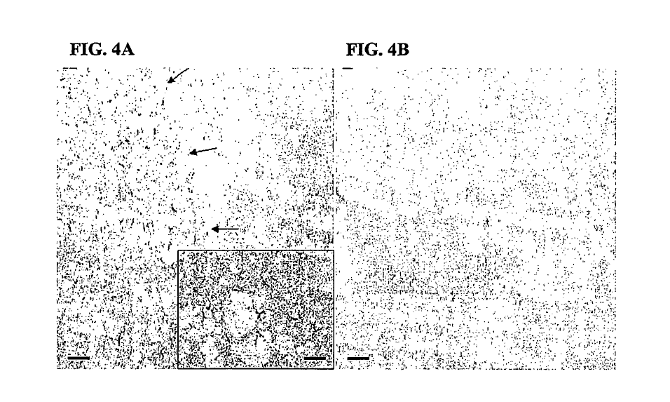

FIG. 4 shows the immunostaining of CB₂ receptors in the caudate-putamen of rats with unilateral injections of malonate. Left panel shows the lesioned side, whereas the right panel displays the non-lesioned side. Note the microglial-like appearance of CB₂ positive cells (insert in A) as well as the spatial segregation within the lesioned striatum (arrows).

It was thought that CB₂ receptors were absent from the striatum in adult mammals. This issue was re-examined in this study and no immunoreactivity was found for this receptor subtype in the intact adult rat using classic immunohistochemical staining methods (FIG. 4). However, it cannot be ruled out that CB₂ receptors may be in fact present in the intact striatum, although at very low levels of expression, as previously reported for the cerebellum (Nuñez E. et al., Synapse 53: 208-13, 2004).

Next, the hypothesis that the expression of the CB₂ receptors could be up-regulated by the malonate-induced neurodegenerative process was tested. Indeed, in contrast to the relative absence of signal for the CB₂ receptor in non-pathological conditions, significant immunoreactivity for the receptor was detected in the lesioned caudate-putamen using classic immunohistochemical staining methods (FIG. 4). The morphology of the cells expressing the CB₂ receptors is characteristic of glial cells, possibly reactive microglia associated to lesioned areas. The presence of CB₂ receptors in a few populations of astrocytes cannot be excluded at this point. The presence on the receptor on microglia is supported by the clear spatial segregation in CB₂ staining that can be observed within the lesioned striatum, with areas affected by malonate administration being strongly positive for CB₂ (FIG. 4). Preliminary co-localization studies tend to confirm the location of CB₂ receptors in glial cells (reactive microglia and/or astrocytes).

These results showing that the presence of CB₂ receptors is dramatically up-regulated in a model of Huntington's disease further support the pivotal role of CB₂ in this disorder.

### Example 6 Potential Mechanisms Involved in Neuroprotection Provided by CB₂ Agonists

The results obtained in previous Examples indicate that the CB₂ receptor and its agonists have a crucial role in neuroprotection. However, as previously explained, cannabinoids, either natural or synthetic, can elicit their physiological effects either through receptor-mediated mechanisms, generally through the cannabinoid receptors, or through non-receptor mediated mechanisms. The potential mechanism(s) by which CB₂ agonists might provide neuroprotection against malonate toxicity, include: (i) a possible increase of endogenous antioxidant defences (antioxidant effect), (ii) the arrest of apoptotic cascade (anti-apoptotic effect), and/or (iii) the reduction of local inflammatory processes (anti-inflammatory effect). Importantly, the malfunctioning of these mechanisms has been related in part to the development of neurodegeneration.

Superoxide dismutase-1 and -2 (SOD-1 and SOD-2) were used as markers of endogenous antioxidant defenses. Their relative levels in lesioned vs. non-lesioned areas of malonate injected rats were measured by in situ hybridization. The protocol used is as described in Example 3. For hybridization, the following synthetic probes of at least 40 nucleotides were used. These probes were selected based on the previously published sequences of rat SOD-1 and SOD-2 (SEQ ID No. 2 and 3, respectively; Kunikowska G. and Jenner P., Brain Res. 922: 51-64, 2001).

SEQ ID No. 2

5′-TCCAGTCTTT GTACTTTCTT CATTTCCACC TTTGCCCAAG
TCATC-3′

SEQ ID No. 3

5′-TGATCTGCGC GTTAATGTGC GGCTCCAGCG CGCCATAGT-3′

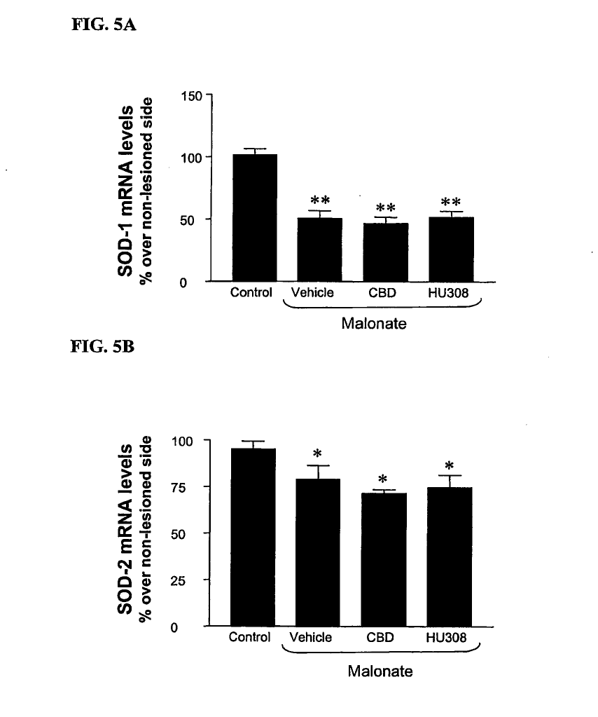

FIG. 5 displays the relative mRNA levels for SOD-1 (Panel A) and SOD-2 (Panel B) in the caudate-putamen of rats with unilateral injections of malonate in animals treated with HU-308 or CBD, and their respective controls of naïve animals (Control) and vehicle treated malonate-lesioned animals (Vehicle). Values correspond to % of the lesioned side over the non-lesioned one for each individual, and are presented as means ±SEM of 5-6 determinations per group. Data were assessed by one-way analysis of variance followed by the Student-Newman-Keuls test (*p<0.05, **p<0.001 vs. the controls).

SOD-1 and SOD-2 are the two isoforms of one of the key enzymes involved in the endogenous defence against oxidative stress. As shown in FIG. 5, HU-308 was unable to prevent the malonate-induced decrease in endogenous antioxidant defences reflected in the reduction of mRNA levels for SOD-1 (F(3,21)=26.32, p<0.0001) and, to a lesser extent, SOD-2 (F(3,22)=4.13, p<0.05; see FIG. 5). Therefore, it is unlikely that HU-308 acts through intrinsic antioxidant properties or that the activation of CB₂ receptors in this model result in an antioxidant effect.

Without wishing to be bound to any particular theory or hypothesis, the results of the above Examples, taken together, teach that unexpectedly activation of CB₂ receptors, rather than CB₁ receptors or cannabinoid receptor-independent antioxidant properties, is the mechanism mediating the cannabinoid-induced neuroprotection in this rat model. This can be concluded by three complementary observations: (i) the effects of selective CB₂ receptor agonists, (ii) the reversion of these effects with CB₂ receptor antagonists, and (iii) the possible occurrence of CB₂ receptor induction in an environment of neuronal damage.

Concerning the first evidence, it has now been disclosed for the first time that HU-308, a selective CB₂ receptor agonist, reduced the malonate-induced deficits in GABA and dopamine and in gene expression of neuronal-specific enolase observed in the caudate-putamen and other basal ganglia. Importantly, these effects were not observed after administration of the CB₁ receptor agonist ACEA or the antioxidant cannabinoid CBD. An important aspect of the effect exerted by HU-308 is that it was produced only in an environment of neuronal damage, since the compound did not alter GABA and dopamine transmission in the non-lesioned side. This observation is certainly related to the fact that CB₂ receptors are not present in the non-lesioned side, but are induced as a consequence of the malonate-induced lesion.

Additional evidence comes from the experiments with a CB₂ receptor antagonist. As detailed above, the effect exerted by HU-308 against the GABA depletion induced by malonate was abolished when this selective CB₂ receptor agonist was co-administered with the selective CB₂ receptor antagonist, SR144528. This observation, taken together with the lack of relevant effects after the administration of CBD, supports a major role for the CB₂ receptors in comparison with additional mechanisms, such as the antioxidant and receptor-independent properties of certain cannabinoid agonists. Though non-receptor mediated mechanisms might in theory represent valid explanations to neuroprotective activity against malonate toxicity, the complete reversion by SR144528 of the neuroprotective effect of HU-308 in this model of HD tells in favor of CB₂ receptor mediation of these effects and against the involvement of significant cannabinoid receptor independent properties.

The pivotal role of CB₂ receptors in the neuroprotective effects of HU-308 is also supported by the observation that malonate-induced neuronal damage leads to up-regulation of the CB₂ receptor subtype. The physiological role of this up-regulatory response during striatal degeneration remains unclear. One possibility is that it may represent part of an endogenous response against the neuronal degeneration. The CB₂ receptor is mostly absent from the brain in normal conditions. The present experiments provide immunohistochemical evidence that indeed CB₂ receptors are scarcely expressed in the striatum of control rats or in the contralateral non-lesioned striatum of malonate-treated rats, but that their expression is up-regulated in the lesioned side. Analysis of the morphological characteristics of cells expressing the CB₂ receptor in the lesioned striatum suggest that this subtype is possibly located in reactive microglial cells, and may be in specific subpopulations of astrocytes.

It should be noted that the present invention is not limited to a particular theory or hypothesis with regards to the mechanism of action by which the CB₂ receptor agonists exert their therapeutic effect.

The present experiments show that CB₂ receptors in the striatum have increased expression in response to neurodegeneration produced by a mitochondrial toxin, and that activation of these receptors by CB₂ selective agonist produces significant neuroprotective effects. These results support the unexpected discovery that CB₂ receptors represent a potential therapeutic target to slow the progression of degeneration in HD, and possibly other neurodegenerative disorders, and that CB₂ agonists finds unexpected and surprising new use against this pathology.

To the extent necessary to understand or complete the disclosure of the present invention, all publications, patents, and patent applications mentioned herein are expressly incorporated by reference in their entirety by reference as is fully set forth herein.

Although the present invention has been described with respect to various specific embodiments presented thereof for the sake of illustration only, such specifically disclosed embodiments should not be considered limiting. Many other such embodiments will occur to those skilled in the art based upon applicants' disclosure herein, and applicants propose to be bound only by the spirit and scone of their invention as defined in the appended claims.

What is claimed is:

1. A method for treating or alleviating Huntington's disease, comprising administering to an individual in need thereof a prophylactically or therapeutically effective amount of a pharmaceutical composition comprising as an active ingredient a CB₂ selective agonist or an isomer, pharmaceutically acceptable salt, ester, polymorph, solvate or prodrug thereof.

2. The method of claim 1, wherein the CB₂ selective agonist is selected from the group consisting of an aminoalkylindole, an anandamide, a 3-aroylindole, an aryl or heteroaryl sulfonate, an arylsulphonamide, a benzamide, a biphenyl-like cannabinoid, a cannabinoid optionally further substituted by one or more fused or bridged mono- or polycyclic rings, a pyrazole-4-carboxamide, an eicosanoid, a dihydroisoindolone, a dihydrooxazole, a α-pinene derivative, a quinazolinedione, a quinolinecarboxylic acid amide, a resorcinol derivative, a tetrazine, a triazine, a pyridazine and a pyrimidine derivative, and isomers, pharmaceutically acceptable salts, esters, polymorphs, solvates and prodrugs thereof.

3. The method of claim 2, wherein the CB₂ selective agonist is a (+)-α-pinene derivative of formula (I): 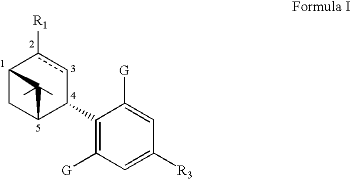
having a specific stereochemistry wherein C-5 is in the (S) configuration, the protons at C-1 and C-5 are cis in relation to one another and the protons at C-4 and C-5 are trans in relation to one another, wherein:
the dashed line between C-2 and C-3 designates an optional double bond;
R₁ is selected from the group consisting of:
(a) —R′ wherein R′ is a C₁-C₅ straight or branched chain alkyl;
(b) —OR″ wherein R″ is a hydrogen or a C₁-C₅ straight or branched chain alkyl optionally containing a terminal —OR′″ or —OC(O)R′″ moiety, wherein R′″ is a hydrogen or a C₁-C₅ straight or branched chain alkyl;
(c) -LN(R″)₂ wherein L is a C₁-C₅ straight or branched chain alkylene and at each occurrence R″ is as previously defined;
(d) -LX wherein L is as previously defined and X is halogen;
(e) -L^{a}C(O)N(R″)₂ wherein La is a direct bond or a C₁-C₅ straight or branched chain alkylene and R″ is as previously defined;
(f) -L^{a}C(O)OR″ or -L^{a}OC(O)R″ wherein La and R″ are as previously defined; and
(g) -LOR′″ wherein L and R′″ are as previously defined;
G is at each occurrence independently selected from the group consisting of hydrogen, halogen and —OR₂ wherein R₂ is a hydrogen or C₁-C₅ straight or branched chain alkyl optionally containing a terminal —OR′″, —OC(O)R′″, C(O)OR′″, or —C(O)R′″ moiety wherein R′″ is as previously defined; and
R₃ is selected from the group consisting of
(a) a C₁-C₁₂ straight or branched chain alkyl;
(b) —OR″″ wherein R″″ is a straight or branched chain C₂-C₈ alkyl which can be optionally substituted at the terminal carbon atom by a phenyl group; and
(c) —(CH₂)ₙOR′″ wherein n is an integer of 1 to 7 and R′″ is as previously defined;
and pharmaceutically acceptable salts, esters, solvates, polymorphs or prodrugs of the compound.

4. The method of claim 3, wherein the CB₂ selective agonist is a compound of formula (I) wherein R₁ is CH₂OH, G is OCH₃, R₃ is 1,1-dimethylheptyl and the dashed line between C-2 and C-3 designates a double bond.

5. The method of claim 1, wherein the pharmaceutical composition further comprises a pharmaceutically acceptable diluent, carrier or excipient.

6. The method of claim 5, wherein the diluent comprises an aqueous solution comprising a pharmaceutically acceptable cosolvent, a micellar solution prepared with natural or synthetic ionic or non-ionic surfactants, or a combination of such cosolvent and micellar solution.

7. The method of claim 6, wherein the cosolvent solution comprises a solution of ethanol, a surfactant and water.

8. The method of claim 5, wherein the carrier is an emulsion comprising a triglyceride, lecithin, an emulsifier, and water.

9. The method of claim 1, wherein the pharmaceutical composition is in a form suitable for oral, parenteral, intravenous, intramuscular, intraperitoneal, subcutaneous, transdermal, intrathecal, rectal or intranasal administration.

10. The method of claim 2, wherein the pharmaceutical composition is in a form suitable for oral, parenteral, intravenous, intramuscular, intraperitoneal, subcutaneous, transdermal, intrathecal, rectal or intranasal administration.

11. The method of claim 3, wherein the pharmaceutical composition is in a form suitable for oral, parenteral, intravenous, intramuscular, intraperitoneal, subcutaneous, transdermal, intrathecal, rectal or intranasal administration.

12. The method of claim 4, wherein the pharmaceutical composition is in a form suitable for oral, parenteral, intravenous, intramuscular, intraperitoneal, subcutaneous, transdermal, intrathecal, rectal or intranasal administration.

13. The method of claim 1, wherein the daily dosage of the CB₂ selective agonist is between 0.01 and 50 mg/kg.

14. A method for preparing a medicament for treating or alleviating Huntington's disease which comprises incorporating a CB₂ selective agonist, or a pharmaceutically acceptable salt, ester, polymorph, solvate or prodrug thereof, into a pharmaceutical composition for use as a medicament for treating or alleviating Huntington's disease.

15. The method of claim 14 wherein the CB₂ selective agonist is selected from the group consisting of an aminoalkylindole, an anandamide, a 3-aroylindole, an aryl or heteroaryl sulfonate, an arylsulphonamide, a benzamide, a biphenyl-like cannabinoid, a cannabinoid optionally further substituted by fused or bridged mono- or polycyclic rings, a pyrazole-4-carboxamide, an eicosanoid, a dihydroisoindolone, a dihydrooxazole, a α-pinene derivatives, a quinazolinedione, a quinolinecarboxylic acid amide, a resorcinol derivative, a tetrazine, a triazine, a pyridazine and a pyrimidine derivative, and pharmaceutically acceptable salts, esters, polymorphs, solvates and prodrugs thereof.

16. The method of claim 15 wherein the CB₂ selective agonist is a (+)-α-pinene derivative of formula (I): 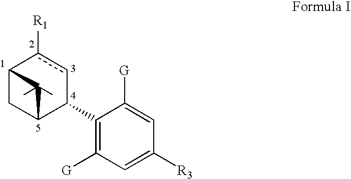
having a specific stereochemistry wherein C-5 is in the (S) configuration, the protons at C-1 and C-5 are cis in relation to one another and the protons at C-4 and C-5 are trans in relation to one another, wherein:
the dashed line between C-2 and C-3 designates an optional double bond;
R₁ is selected from the group consisting of:
(a) —R′ wherein R′ is a C₁-C₅ straight or branched chain alkyl;
(b) —OR″ wherein R″ is a hydrogen or a C₁-C₅ straight or branched chain alkyl optionally containing a terminal —OR′″ or —OC(O)R′″ moiety, wherein R′″ is a hydrogen or a C₁-C₅ straight or branched chain alkyl;
(c) -LN(R″)₂ wherein L is a C₁-C₅ straight or branched chain alkylene and at each occurrence R″ is as previously defined;
(d) -LX wherein L is as previously defined and X is halogen;
(e) -L^{a}C(O)N(R″)₂ wherein La is a direct bond or a C₁-C₅ straight or branched chain alkylene and R″ is as previously defined;
(f) -L^{a}C(O)OR″ or -L^{a}OC(O)R″ wherein La and R″ are as previously defined; and
(g) -LOR′″ wherein L and R′″ are as previously defined;
G is at each occurrence independently selected from the group consisting of hydrogen, halogen and —OR₂ wherein R₂ is a hydrogen or C₁-C₅ straight or branched chain alkyl optionally containing a terminal —OR′″, —OC(O)R′″, C(O)OR′″, or —C(O)R′″ moiety wherein R′″ is as previously defined; and
R₃ is selected from the group consisting of
(a) a C₁-C₁₂ straight or branched chain alkyl;
(b) —OR″″ wherein R″″ is a straight or branched chain C₂-C₈ alkyl which can be optionally substituted at the terminal carbon atom by a phenyl group; and
(c) —(CH₂)ₙOR′″ wherein n is an integer of 1 to 7 and R′″ is as previously defined;
and pharmaceutically acceptable salts, esters, solvates, polymorphs or prodrugs of the compound.

17. The method of claim 16, wherein the CB₂ selective agonist is a compound of formula (I) wherein R₁ is CH₂OH, G is OCH₃, R₃ is 1,1-dimethylheptyl and the dashed line between C-2 and C-3 designates a double bond.

18. The method of claim 14, wherein the medicament further comprises a pharmaceutically acceptable diluent, carrier or excipient.

19. The method of claim 18, wherein the diluent comprises an aqueous solution comprising a pharmaceutically acceptable cosolvent, a micellar solution prepared with natural or synthetic ionic or non-ionic surfactants, or a combination of such cosolvent and micellar solutions.

20. The method of claim 19, wherein the cosolvent solution comprises a solution of ethanol, a surfactant and water.

21. The method of claim 18, wherein the carrier is an emulsion comprising triglycerides, lecithin, an emulsifier, and water.

22. The method of claim 14, wherein the medicament is in a form suitable for oral, parenteral, intravenous, intramuscular, intraperitoneal, subcutaneous, transdermal, intrathecal, rectal or intranasal administration.0

23. The method of claim 14, wherein the daily dosage of the CB₂ selective agonist is between 0.01 and 50 mg/kg.
